(19) United States
(12) Patent Application Publication
(10) Pub. No.: US 2025/0305993 A1
(43) Pub. Date: Oct. 2, 2025

(54) ULTRASONIC PHASED ARRAY SYSTEM BASED ON A METHOD FOR INTELLIGENT PLANNING OF TARGET PARAMETERS

(71) Applicant: NANJING KHONS MEDTECH CO., LTD., Nanjing (CN)

(72) Inventors: Xiangqing KONG, Nanjing (CN); Enguang ZHANG, Nanjing (CN); Ruibiao GUO, Nanjing (CN); Jing CAI, Nanjing (CN); Feifei CHE, Nanjing (CN); Zhichao FU, Nanjing (CN)

(73) Assignee: NANJING KHONS MEDTECH CO., LTD., Nanjing (CN)

(21) Appl. No.: 18/806,114

(22) Filed: Aug. 15, 2024

(30) Foreign Application Priority Data
Mar. 28, 2024 (CN) 202410366009.8

(51) Int. Cl. G01N 29/26; G01N 29/24

(52) U.S. Cl. CPC G01N 29/262; G01N 29/24; G01N 2291/106

(57) ABSTRACT

An ultrasonic phased array system based on a method for intelligent planning of target parameters includes a central control unit, an ultrasonic imaging unit, a phased array emission unit, a mechanical motion unit, a degassed water treatment unit and a composite probe. The central control unit controls each unit and plans target coordinates and emission parameters in a target region. The ultrasonic imaging unit acquires image data of the target region. The phased array emission unit generates one or more focal points in the target region and can control the focusing positions of the focal points. The mechanical motion unit moves the composite probe. The degassed water treatment unit generates degassed water and controls its circulation.

### TECHNICAL FIELD

The present invention relates to an ultrasonic phased array system, particularly to an ultrasonic phased array system based on a method for intelligent planning of target parameters, and pertains to the field of biomedical instruments and devices.

### BACKGROUND ART

High-intensity focused ultrasound (HIFU) technology is a new technology by which power ultrasound is emitted from outside to inside the body and converges in a specific target region, thereby producing a certain biological effect on human body and achieving a non-invasive therapeutic effect.

The existing HIFU devices mainly include single-element phased array devices and multi-element phased array devices. In such phased array focused ultrasound devices, the size of the focal region in the geometric focal position is typically used as a common standard for all spatial positions, and a sequence of focal points in a treatment target region is planned on this basis. However, the reality is that phased array focused ultrasound devices have different focal region sizes in different spatial positions. Although the planning method of setting the focal region size as a static value is simple in design, it results in inconsistent distribution of focal regions in different positions of the treatment target region. Particularly, in new indication application scenarios, such as visceral fat modification and renal sympathetic nerve ablation, higher requirements are put forward for target region planning precision of the phased array focused ultrasound system due to the presence of important organs and tissues adjacent to the target region. Therefore, it is necessary to fully consider the changes of focal region size in different spatial positions during planning of a sequence of focal points.

Further, in the ultrasonic phased array system, the means for real-time tracking are inadequate when the focal position changes. The current system lacks an effective mechanism to dynamically track and monitor changes in focal position, thereby affecting the accuracy and safety of treatment. In order to make up for this shortcoming, it is necessary to introduce a technology that can realize real-time tracking of a focal position.

### SUMMARY OF THE INVENTION

The objective of the present invention is to overcome the shortcoming of the prior art and provide an ultrasonic phased array system based on a method for intelligent planning of target parameters.

In order to achieve the foregoing objective, the present invention adopts the following technical solution: An ultrasonic phased array system based on a method for intelligent planning of target parameters, comprising a central control unit, an ultrasonic imaging unit, a phased array emission unit, a mechanical motion unit, a degassed water treatment unit and a composite probe, wherein:

The central control unit comprises one or more processors, which are used for controlling the ultrasonic imaging unit, the phased array emission unit, the mechanical motion unit, the degassed water treatment unit and the composite probe and for planning target coordinates and emission parameters in a target region;

The ultrasonic imaging unit acquires image data of the target region through an ultrasonic imaging probe in the composite probe;

The phased array emission unit generates one or more focal points in the target region by independently controlling emission phases of different array elements of a phased array transducer and can control focusing positions of one or more focal points;

The mechanical motion unit comprises a mechanical motion driver and a multi-dimensional motion mechanical structure, and is used for moving the composite probe;

The degassed water treatment unit is used for generating degassed water, transmitting the degassed water to a water tank of the composite probe and controlling the degassed water to circulate between the water tank and the degassed water treatment unit;

The composite probe comprises a phased array transducer, an ultrasonic imaging probe and an information storage device.

Further, the central control unit receives the following information set by a user: spatial peak time average sound intensity Iₛₚₜₐ, ultrasonic irradiation time t and temperature threshold Tₚ of the focal region.

Further, the image data acquired by the ultrasonic imaging unit includes grayscale image data and color image data; at the same time, ultrasonic RF data of the target region is also acquired, which is an original ultrasonic echo signal after beamforming; the ultrasonic imaging unit sends a frame pulse signal to the phased array emission unit when a first line of each frame of ultrasonic image starts scanning, and sends a line pulse signal to the phased array emission unit when each line of each frame of ultrasonic image except the first line starts scanning.

Further, the phased array emission unit controls the phased array transducer to emit ultrasound and counts line pulse signals sent by the ultrasonic imaging unit when receiving a frame pulse signal from the ultrasonic imaging unit; stops the phased array transducer sending ultrasound and counts line pulse signals again when the count reaches a first set threshold A; continues to control the phased array transducer to emit ultrasound when the count reaches a second set threshold B; repeats the above process when receiving a frame pulse signal from the ultrasonic imaging unit again; by controlling the first set threshold and the second set threshold, the phased array transducer is stopped emitting ultrasound when the ultrasonic imaging unit scans ultrasonic images in the target region, thereby avoiding the interference of high-intensity ultrasound on weak ultrasonic echo signals in the target region.

Further, the information storage device is used for storing various parameters of the phased array transducer;

The phased array transducer consists of two or more independent array elements;

The ultrasonic imaging probe is coaxially assembled with the center of the phased array transducer. The ultrasonic imaging probe is a 2D ultrasonic imaging probe or a 3D ultrasonic imaging probe.

Further, the ultrasonic imaging probe is a 2D ultrasonic imaging probe, and the composite probe further comprises a position control device; the position control device controls the 2D ultrasonic imaging probe to rotate along the central axis according to the position of focal point coordinates, and has the plane where the imaging field of the 2D ultrasonic imaging probe is located pass through the focal point coordinates all the time so as to realize real-time display of a focal position through an ultrasonic image in the process of ultrasonic emission.

Still further, when the target coordinates are (xᵢ, yⱼ,zₖ), the position control device controls the ultrasonic imaging probe to rotate along the central axis at an angle of a, which is calculated according to the following formula:

$$\{ \begin{matrix}α = arctan ( \frac{{y}_{j}}{{x}_{i}} ) , {x}_{i} > 0 , {y}_{j} > 0 \\ α = π + arctan ( \frac{{y}_{j}}{{x}_{i}} ) , {x}_{i} < 0 \\ α = 2 π + arctan ( \frac{{y}_{j}}{{x}_{i}} ) , {x}_{i} > 0 , {y}_{j} < 0\end{matrix} .$$

Further, the central control unit performs intelligent planning of target coordinates in the target region according to different focal region sizes when the focal point is in different spatial positions; the method for intelligent planning of target coordinates in the target region comprises the following steps:

S001: setting the center of the top surface of the phased array transducer as an origin of a coordinate system, the axis direction of the phased array transducer as Z axis, the imaging scanning direction of the imaging probe as X axis, and the direction perpendicular to the imaging scanning direction of the imaging probe as Y axis;

S002: obtaining a coordinate set of pixels of the target region boundary set by a user;

S003: calculating according to the coordinate set of pixels and the distance between pixels dₚᵢₓₑₗ to obtain: physical coordinates of the target region boundary Sₚ=(xₛₚ,yₛₚ, zₛₚ),p∈1, 2, . . . ,P;

S004: obtaining the following built-in information of the system: safety distance sdₓsd_{y} sd_{z} between focal region boundary and target region boundary in X, Y, Z axis;
obtaining the following information set by the user: distance dₓ, d_{y}, d_{z} between focal region boundaries in X, Y, Z direction;
obtaining the following information set by the user: spatial peak time average sound intensity Iₛₚₜₐ, ultrasonic irradiation time t and temperature threshold Tₚ of the focal region;

S005: expanding the target region boundary Sₚ into a rectangular area S′ₚ;
wherein, an upper surface and a lower surface of the rectangular area S′ₚ are perpendicular to Z axis and pass through points (0,0,min(zₛₚ)) and (0,0,max(zₛₚ)) respectively; a left surface and a right surface are perpendicular to X axis and pass through points (min(xₛₚ),0,0) and (max(xₛₚ),0,0), respectively; and a front surface and rear surface are perpendicular to Y axis and pass through points (0,min(yₛₚ),0) and (0,max(yₛₚ),0), respectively;

S006: setting target coordinates within the rectangular area S′ₚ to:

(xᵢ′,yⱼ′,zₖ′),i∈1,2, . . . ,1;j∈1,2, . . . ,J;k∈1,2, . . . ,K;
where, the point (xᵢ′,yⱼ′, zₖ′) is located in the X-Y plane which passes through a point (0,0, zₖ′) and is perpendicular to Z axis;

S007: calculating: coordinates (zₖ′),k∈1, 2, . . . , K of the target (xᵢ′,yⱼ′, zₖ′) on Z axis, i.e., the Z axis coordinates that a different X-Y plane where the target is located passes, coordinates (xᵢ′), i∈1,2, . . . ,I of the target (xᵢ′,yⱼ′, zₖ′) on X axis, and coordinates (yⱼ′), j∈ 1,2, . . . ,J of the target (xᵢ′,yⱼ′, zₖ′) on Y axis;

S008: calculating according to a target region boundary Sₚ=(xₛₚ, yₛₚ, zₛₚ), p∈1, 2, . . . , P and a safety distance sdₓ, sd_{y}, sd_{z} to obtain a safety boundary Saₚ=(xₛₐₚ, yₛₐₚ, zₛₐₚ), p∈1, 2, . . . , P;

S009: for coordinates of each focal point in (xᵢ′,yⱼ′, zₖ′), obtaining, through the following calculation, new coordinate points corresponding to the limit positions of the focal region boundary on X, Y and Z axes:

$$( {x}_{i}^{′} \pm \frac{{WX}_{0}}{2} , {y}_{j}^{′} , {z}_{k}^{′} ) , ( {x}_{i}^{′} , {y}_{j}^{′} \pm \frac{{WY}_{0}}{2} , {z}_{k}^{′} ) , ( {x}_{i}^{′} , {y}_{j}^{′} , {z}_{k}^{′} \pm \frac{{L}_{k}}{2} ) ;$$

S010: further determining by the ray method whether the new coordinate points are within the safety boundary Saₚ=(xₛₐₚ, yₛₐₚ, zₛₐₚ), and if any new coordinate point is not within the safety boundary, then deleting the corresponding focal point coordinates of the new coordinate point in (xᵢ′,yⱼ′, zₖ′);

S011: repeating S009 and S010 until the coordinates of each focal point in (xᵢ′,yⱼ′, zₖ′) are traversed and final target coordinates (xᵢ, yⱼ, zₖ) are obtained.

Still further, calculating coordinates (zₖ′),k∈ 1, 2, . . . , K of the target (xᵢ′,yⱼ′, zₖ′) on Z axis through the following steps:
a) calculating first coordinates z₁′: z₁′=min(zₛₚ)+d_{z}+Δz×n, where n≥1, n is an integer; n is added with 1 step by step, starting from 1, to perform the following calculation:
calculating focal region length L, focal region width WX of X axis, and focal region width WY of Y axis by the focal region calculation method of sound field when the focal point is located at (0,0, min(zₛₚ)+d_{z}+Δz×n), recording min(zₛₚ)+d_{z}+Δ z×n as z₁′ when Formula

$$\frac{L}{2} \geq Δ z \times n$$

is satisfied for the first time, i.e., coordinates on Z axis that the first X-Y plane where the target is located passes, and now recording the focal region length as L₁, focal region width of X axis as WX₁, and focal region width of Y axis as WY₁;
where, Δz is a step threshold when the coordinates of the target on Z axis are calculated;
b) calculating m^{th} coordinates zₘ′:

$${z}_{m}^{′} = {z}_{m - 1}^{′} + \frac{{L}_{m - 1}}{2} + {d}_{z} + Δ z \times n ,$$

where n≥1, n is an integer, m≥2, m is an integer; n is added with 1 step by step, starting from 1, to perform the following calculation:
calculating focal region length L, focal region width WX of X axis, and focal region width WY of Y axis by the focal region calculation method of sound field when the focal point is located at

$$( 0 , 0 , {z}_{m - 1}^{′} + \frac{{L}_{m - 1}}{2} + {d}_{z} + Δ z \times n ) ,$$

recording

$${z}_{m - 1}^{′} + \frac{{L}_{m - 1}}{2} + {d}_{z} + Δ z \times n as {z}_{m}^{′}$$

when Formula

$$\frac{{L}_{m}}{2} \geq Δ z \times n$$

is satisfied for the first time, and

$$\frac{{L}_{m}}{2} \leq ( max ( {z}_{s p} ) - {d}_{z} ) ,$$

i.e., coordinates on Z axis that the m^{th} X-Y plane where the target is located passes, and now recording the focal region length as Lₘ, focal region width of X axis as WXₘ, and focal region width of Y axis as WYₘ;
c) adding 1 to m, then repeating step b), stopping calculation when

$$\frac{{L}_{m}}{2} > ( max ( {z}_{sp} ) - {d}_{z} )$$

appears for the first time, recording the result

$${z}_{m - 1}^{′} + \frac{{L}_{m - 1}}{2} + {d}_{z} + Δ z \times n$$

of the calculation before this calculation as z_{K}′, i.e., coordinates on Z axis that the K^{th} X-Y plane where the target is located passes, and now recording the focal region length as L_{K}, focal region width of X axis as WX_{K}, and focal region width of Y axis as WY_{K}, where K=m−1;
d) obtaining from the above step the coordinates (zₖ′) of the target (xᵢ′,yⱼ′, zₖ′)on Z axis, focal region length (Lₖ), focal region width of X axis (WXₖ), and focal region width of Y axis (WYₖ),k∈ 1, 2, . . . , K.

Still further, calculating coordinates (xᵢ′), i∈1,2, . . . ,I of target (xᵢ′, yⱼ,zₖ′) on X axis through the following steps:
a) calculating I′ according to Formula min(xₛₚ)+dₓ×I′+WX₀×I′+dₓ≤max(xₛₚ), and recording the integer part of I′ as I;
b) calculating xᵢ′ according to the following formula:

$${x}_{i}^{′} = min ( {x}_{sp} ) + {d}_{x} \times i + {WX}_{0} \times i - \frac{{WX}_{0}}{2} , i ∈ 1 , 2 , … , I .$$

Still further, calculating coordinates (yⱼ′), j∈1,2, . . . ,J of the target (xᵢ′,yⱼ′, zₖ′) on y axis through the following steps:
a) calculating J′ according to Formula min(yₛₚ)+d_{y}×J′+WY₀×J′+d_{y}≤max(yₛₚ), and recording the integer part of J′ as J;
b) calculating yⱼ′ according to the following formula:

$${y}_{j}^{′} = min ( {y}_{sp} ) + {d}_{y} \times j + {WY}_{0} \times j - \frac{{WY}_{0}}{2} , j ∈ 1 , 2 , … , J .$$

Further, the central control unit performs intelligent planning of target emission parameters in the target region according to different focal region sizes when the focal point is in different spatial positions; the method for intelligent planning of target emission parameters comprises the following steps:

S101: calculating focal region sound power P of different focal point coordinates according to focal region length Lₖ of target coordinates, focal region width of X axis WXₖ, focal region width of Y axis WYₖ and set spatial peak time average sound intensity Iₛₚₜₐ:

$${P}_{un} = {I}_{spta} \times π \times {( 0.25 ( {WX}_{k} + {WY}_{k} ) )}^{2} ;$$

S102: calculating emission power P_{em,n} of each array element at different focal point coordinates according to the power weight Qₘ and electroacoustic conversion efficiency Kₘ of each array element of the phased array transducer:

$${P}_{em , n} = \frac{{P}_{un}}{{K}_{m} {\sum }_{m = 1}^{M} {Q}_{m}} , m = 1 , 2 , … , M ; n = 1 , 2 , … , N ;$$

S103: calculating emission signal voltage (U_{m,n}) of each array element at different focal point coordinates according to the impedance characteristic (Zₘ),m=1,2, . . . ,M of each array element of the phased array transducer:

$${U}_{m , n} = \sqrt{\frac{{P}_{em , n} {Z}_{m}}{\frac{B}{C}}} , m = 1 , 2 , … , M ; n = 1 , 2 , … , N ;$$

where, C is the number of scanning lines needed by the ultrasonic imaging unit to scan a frame of ultrasonic image;

S104: calculating the maximum value of (U_{m,n}), recording it as U₀, and setting U₀ as a supply voltage of a power amplifier circuit in the phased array emission unit;

S105: calculating emission signal duty cycle D_{m,n} of each array element of the phased array transducer according to U_{m,n} and U₀:

$${D}_{m , n} = 0.5 \sqrt{\frac{{U}_{m , n}}{{U}_{0}}} .$$

Compared with the prior art, the present invention has the following advantages:
1) The present invent achieves intelligent planning of target coordinates in the target region according to different focal region sizes when the focal point is in different spatial positions, can significantly improve the focal region planning precision of the ultrasonic phased array system, and improves the accuracy and safety of the device during use.
2) The present invent achieves intelligent planning of target emission parameters in the target region according to different focal region sizes when the focal point is in different spatial positions, and realizes consistency of spatial peak time average sound intensity Iₛₚₜₐeven under different focal region sizes.
3) The method for calculating target emission parameters, such as focal region sound power, emission power and emission signal voltage, according to the actual impedance characteristics, electroacoustic conversion efficiency and power weight of each array element of the phased array transducer makes up for element difference in calculation, realizes sound power calibration and improves the accurate control of ultrasonic irradiation energy.

### BRIEF DESCRIPTION OF THE DRAWINGS

FIG. 1 is a schematic view of the present invention, where: 1 is a central control unit; 2 is an ultrasonic imaging unit; 3 is a phased array emission unit; 4 is a mechanical motion unit; 5 is a degassed water treatment unit; 6 is a composite probe; 61 is a position control device, 62 is an ultrasonic imaging probe, 63 is a phased array transducer and 64 is an information storage device.

FIG. 2 shows focal region sizes of a phased array transducer focusing in different spatial positions in this embodiment.

FIG. 3 is a schematic view of intelligent planning of target coordinates.

FIG. 4 is a schematic view of calculation of Z axis coordinates in intelligent planning of target coordinates.

FIG. 5 is a flow diagram of a method for intelligent planning of target coordinates.

FIG. 6 is a flow diagram of a method for intelligent planning of target emission parameters.

FIG. 7 is a structural schematic view of an embodiment of a composite probe.

FIG. 8 is a schematic view of tracking of focal point coordinates, where 100 shows the focal position of a phased array transducer.

### DETAILED DESCRIPTION

Below the present invention is elaborated in conjunction with accompanying drawings and specific embodiments.

FIG. 1 shows an ultrasonic phased array system based on a method for intelligent planning of target parameters, comprising a central control unit 1, an ultrasonic imaging unit 2, a phased array emission unit 3, a mechanical motion unit 4, a degassed water treatment unit 5 and a composite probe 6.

The central control unit 1 comprises one or more processors, which are electrically connected to the ultrasonic imaging unit 2, the phased array emission unit 3, the mechanical motion unit 4, the degassed water treatment unit 5 and the composite probe 6, and controls each unit.

The central control unit 1 can receive the following information set by a user: spatial peak time average sound intensity Iₛₚₜₐ, ultrasonic irradiation time t and temperature threshold Tₚ of the focal region. The present invention can realize consistency of spatial peak time average sound intensity even under different focal region sizes in different spatial positions.

The ultrasonic imaging unit 2 acquires image data of the target region through the ultrasonic imaging probe 62 in the composite probe 6. The image data includes grayscale image data and color image data; at the same time, ultrasonic RF data of the target region is also acquired, which is an original ultrasonic echo signal after beamforming.

The ultrasonic imaging unit 2 sends a frame pulse signal to the phased array emission unit 3 when a first line of each frame of ultrasonic image starts scanning, and sends a line pulse signal to the phased array emission unit 3 when each line of each frame of ultrasonic image except the first line starts scanning. In this embodiment, the number of scanning lines required to scan a frame of ultrasonic image is 128.

The phased array emission unit 3 generates one or more focal points in the target region by independently controlling emission phases of different array elements of a phased array transducer and can control focusing positions of one or more focal points

The phased array emission unit 3 controls the phased array transducer to emit ultrasound and counts line pulse signals sent by the ultrasonic imaging unit 2 when receiving a frame pulse signal from the ultrasonic imaging unit 2; stops the phased array transducer sending ultrasound and counts line pulse signals again when the count reaches a first set threshold A; continues to control the phased array transducer to emit ultrasound when the count reaches a second set threshold B; repeats the above process when receiving a frame pulse signal from the ultrasonic imaging unit 2 again. By controlling the first set threshold and the second set threshold, the phased array transducer can be stopped emitting ultrasound when the ultrasonic imaging unit 2 scans ultrasonic images in the target region, thereby avoiding the interference of high-intensity ultrasound on weak ultrasonic echo signals in the target region. In this embodiment, in order to control the middle 50% area of the ultrasonic image from high-intensity ultrasonic interference, the first set threshold A is set to 32, and the second set threshold B is set to 64.

The mechanical motion unit 4 comprises a mechanical motion driver and a multi-dimensional motion mechanical structure and is used for moving the composite probe 6.

The degassed water treatment unit 5 is used for generating degassed water, transmitting the degassed water to a water tank of the composite probe 6 and controlling the degassed water to circulate between the water tank and the degassed water treatment unit.

The composite probe 6 comprises a phased array transducer 63, an ultrasonic imaging probe 62, a position control device 61 and an information storage device 64.

The information storage device 64 is used for storing the impedance characteristic, power weight, electroacoustic conversion efficiency and other parameters of each array element of the phased array transducer.

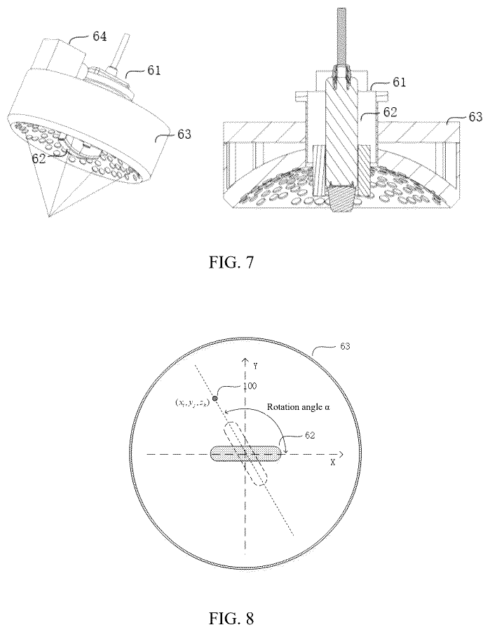

As shown in FIG. 7, the phased array transducer 63 in this embodiment is a domical phased array transducer, and the number of array elements is 190.

As shown in FIG. 7 and FIG. 8, the ultrasonic imaging probe 62 is coaxially assembled with the center of the phased array transducer 63. In this embodiment, the ultrasonic imaging probe 62 is a 2D ultrasonic imaging probe.

As shown in FIG. 7 and FIG. 8, in a solution where the ultrasonic imaging probe 62 is a 2D ultrasonic imaging probe, the composite probe 6 further comprises a position control device 61. The position control device 61 controls the 2D ultrasonic imaging probe to rotate along the central axis according to the position of focal point coordinates, and has the plane where the imaging field of the 2D ultrasonic imaging probe is located pass through the focal point coordinates all the time so as to realize real-time display of a focal position through an ultrasonic image in the process of ultrasonic emission. As shown in FIG. 8, in this embodiment, when the target coordinates are (xᵢ, yⱼ,zₖ), the position control device 61 controls the ultrasonic imaging probe 62 to rotate along the central axis at an angle of a, which is calculated according to the following formula:

$$\{ \begin{matrix}α = arctan ( \frac{{y}_{j}}{{x}_{i}} ) , {x}_{i} > 0 , {y}_{j} > 0 \\ α = π + arctan ( \frac{{y}_{j}}{{x}_{i}} ) , {x}_{i} < 0 \\ α = 2 π + arctan ( \frac{{y}_{j}}{{x}_{i}} ) , {x}_{i} > 0 , {y}_{j} < 0\end{matrix} .$$

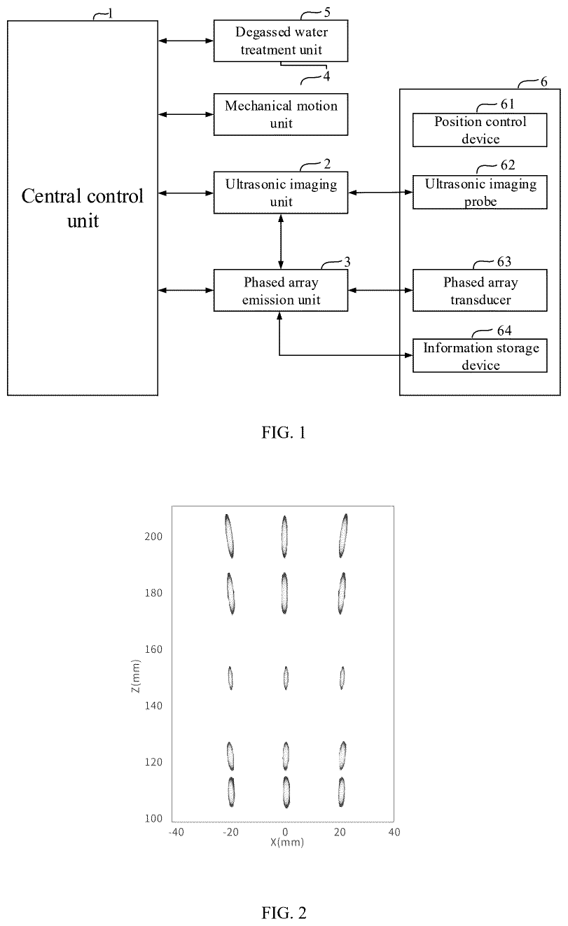

As shown in FIG. 2, the central control unit 1 performs intelligent planning of target coordinates in the target region according to different focal region sizes when the focal point is in different spatial positions. In a phased array focused ultrasound system, focal region parameters, such as focal region length, focal region width and focal region deflection angle, are different when a focal point is in different spatial positions. Therefore, when calculating target coordinates in a target region, calculating the target coordinates according to actual focal region parameters can make target distribution more accurate, effective and secure. However, in practical engineering applications, if all focal region parameters are taken into account in the calculation process, the design of the computing system will be too complicated, and the computing power required will be very high, which will greatly reduce actual availability. FIG. 2 shows focal region sizes of a phased array transducer focusing in different spatial positions. It can be seen that: 1) The focal region width does not have much change in the same X-Y plane perpendicular to Z axis, so the focal region width can be simplified to a fixed value. Typically, the maximum value within the zooming range of the phased array transducer is selected; 2) The focal region length in the direction of Z axis changes obviously, and in the same X-Y plane perpendicular to Z axis, focal region length does not have much change. Therefore, the same value can be used for focal region length in the same X-Y plane perpendicular to Z axis. The focal region length when the focal point is on the axis of the phased array transducer is selected typically, which greatly reduces computing complexity.

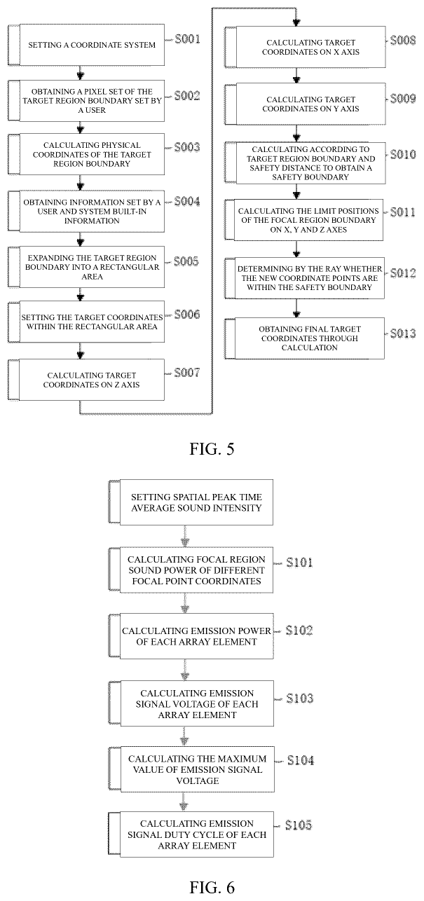

In this embodiment, as shown in FIG. 5, the method for intelligent planning of target coordinates in the target region comprises the following steps:

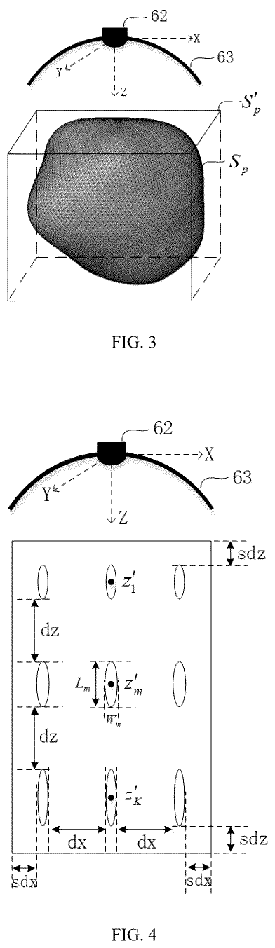

S001: as shown in FIG. 3, setting the center of the top surface of the phased array transducer as an origin of a coordinate system, the axis direction of the phased array transducer as Z axis, the imaging scanning direction of the imaging probe as X axis, and the direction perpendicular to the imaging scanning direction of the imaging probe as Y axis.

S002: as shown in FIG. 3, obtaining a coordinate set of pixels of the target region boundary set by a user, wherein the pixels refer to the pixels of grayscale image data or color image data obtained by the ultrasonic imaging unit 2.

In this embodiment, the size of each frame of ultrasonic image data is 640 (W)*680 (H), so the distance between pixels dₚᵢₓₑₗ is equal to the current depth of ultrasonic imaging divided by 680.

S003: calculating according to the coordinate set of pixels and the distance between pixels dₚᵢₓₑₗ to obtain: physical coordinates of the target region boundary “Sₚ=(xₛₚ, yₛₚ, zₛₚ), p∈1, 2, . . . ,P.

S004: further, obtaining the following built-in information of the system: safety distance sdₓ, sd_{y}, sd_{z} between focal region boundary and target region boundary in X, Y, Z axis.

In practical applications, the distribution positions of a focal point and a focal region in a tissue deviates from the positions measured through sound field simulation and in a sound field test system. Therefore, for safety consideration, normally, a certain safety distance is set inward from the target region boundary, so as to ensure that the focal region will not offset out of the target region boundary when it is located at the edge of the target region. The value of this safety distance is an empirical value, which varies when the focal point is focused on different biological tissues. Normally, fresh animal tissues can be used for simulated determination.

Further, obtaining the following information set by the user: distance dₓ, d_{y}, d_{z} between focal region boundaries in X, Y, Z direction.

Further, obtaining the following information set by the user: spatial peak time average sound intensity Iₛₚₜₐ, ultrasonic irradiation time t and temperature threshold Tₚ of the focal region.

S005: as shown in FIG. 3, expanding the target region boundary Sₚ into a rectangular area Sₚ′.
wherein, an upper surface and a lower surface of the rectangular area Sₚ′ are perpendicular to Z axis and pass through points (0,0, min(zₛₚ)) and (0,0,max(zₛₚ)) respectively; a left surface and a right surface are perpendicular to X axis and pass through points (min(xₛₚ),0,0) and (max(xₛₚ),0,0) respectively; and a front surface and rear surface are perpendicular to Y axis and pass through points (0, min(yₛₚ),0) and (0,max(yₛₚ),0) respectively.

S006: setting target coordinates within the rectangular area Sₚ′ to:

(xᵢ′,yⱼ′,zₖ′),i∈1,2, . . . ,1;j∈1,2, . . . ,J;k∈1,2, . . . ,K;
where, the point (xᵢ′,yⱼ′, zₖ′) is located in the X-Y plane which passes through a point (0,0, zₖ′) and is perpendicular to Z axis.

S007: as shown in FIG. 1 and FIG. 4, calculating coordinates (zₖ′),k ∈ 1,2, . . . ,K of the target (xᵢ′,yⱼ′, zₖ′) on Z axis, i.e., the Z axis coordinates that a different X-Y plane where the target is located passes.
a) calculating first coordinates z₁′: z₁′=min(zₛₚ)+d_{z}+Δ z×n, where n≥1, n is an integer.
n is added with 1 step by step, starting from 1, to perform the following calculation: calculating focal region length L, focal region width WX of X axis, and focal region width WY of Y axis by the focal region calculation method of sound field when the focal point is located at (0,0, min(zₛₚ)+d_{z}+Δ z×n), recording min(zₛₚ)+d_{z}+Δ z×n as z₁′ when Formula

$$\frac{L}{2} \geq Δ z \times n$$

is satisfied for the first time, i.e., coordinates on Z axis that the first X-Y plane where the target is located passes, and now recording the focal region length as L₁, focal region width of X axis as WX₁, and focal region width of Y axis as WY₁; where, Δz is a step threshold when the coordinates of the target on Z axis are calculated. This value is determined by the calculation accuracy of different focal region lengths expected by the specific embodiment. In this embodiment, Δz=0.1 mm.

In this embodiment, the Rayleigh integral method is used to calculate the focal region of the sound field.
b) calculating m^{th} coordinates zₘ′:

$${z}_{m}^{′} = {z}_{m - 1}^{′} + \frac{{L}_{m - 1}}{2} + {d}_{z} + Δ z \times n ,$$

where n≥1, n is an integer, m≥2, m is an integer.
n is added with 1 step by step, starting from 1, to perform the following calculation: calculating focal region length L, focal region width WX of X axis, and focal region width WY of Y axis by the focal region calculation method of sound field when the focal point is located at

$$( 0 , 0 , {z}_{m - 1}^{′} + \frac{{L}_{m - 1}}{2} + {d}_{z} + Δ z \times n ) ,$$

recording

$${z}_{m - 1}^{′} + \frac{{L}_{m - 1}}{2} + {d}_{z} + Δ z \times n$$

as zₘ′ when Formula

$$\frac{{L}_{m}}{2} \geq Δ z \times n$$

is satisfied for the first time, and

$$\frac{{L}_{m}}{2} \leq ( max ( {z}_{sp} ) - {d}_{z} ) ,$$

i.e., coordinates on Z axis that the m^{th} X-Y plane where the target is located passes, and now recording the focal region length as Lₘ, focal region width of X axis as WXₘ, and focal region width of Y axis as WYₘ.
c) adding 1 to m, then repeating step b), stopping calculation when

$$\frac{{L}_{m}}{2} > ( max ( {z}_{sp} ) - {d}_{z} )$$

appears for the first time, recording the result

$${z}_{m - 1}^{′} + \frac{{L}_{m - 1}}{2} + {d}_{z} + Δ z \times n$$

of the calculation before this calculation as z_{K}′, i.e., coordinates on Z axis that the K^{th} X-Y plane where the target is located passes, and now recording the focal region length as L_{K}, focal region width of X axis as WX_{K}, and focal region width of Y axis as WY_{K}, where K=m−1.
d) obtaining from the above step the coordinates (zₖ′) of the target (xᵢ′,y^{j}′,zₖ′) on Z axis, focal region length (Lₖ), focal region width (WXₖ) of X axis, and focal region width (WYₖ) of Y axis k∈ 1, 2, . . . , K
calculating the maximum value in the focal region width (WXₖ) of X axis, recording it as WX₀, and setting WX₀ as the focal region width of all focal point coordinates.
calculating the maximum value in the focal region width (WYₖ) of Y axis, recording it as WY₀, and setting WY₀ as the focal region width of all focal point coordinates.

S008: calculating coordinates (xᵢ′),i∈1,2, . . . ,I of the target (xᵢ′,y^{j}′,zₖ′)on X axis.
a) calculating I′ according to Formula min(xₛₚ)+dₓ×I′+W₀×I′+dₓ≤max(xₛₚ), and recording the integer part of I′ as I.
b) calculating xᵢ′ according to the following formula:

$${x}_{i}^{′} = min ( {x}_{sp} ) + {d}_{x} \times i + {WX}_{0} \times i - \frac{{WX}_{0}}{2} , j ∈ 1 , 2 , … , I$$

S009: calculating coordinates (yⱼ′), j∈1, 2, . . . , J of the target (xᵢ′,y^{j}′,zₖ′) on Y axis.
a) calculating J′ according to Formula min(yₛₚ)+d_{y}×J′+W₀×J′+d_{y}≤max(yₛₚ), and recording the integer part of J′ as J.
b) calculating yⱼ′ according to the following formula:

$${y}_{j}^{′} = min ( {y}_{sp} ) + {d}_{y} \times j + {WY}_{0} \times j - \frac{{WY}_{0}}{2} , j ∈ 1 , 2 , … , J .$$

S010: calculating according to a target region boundary Sₚ=(xₛₚ, yₛₚ,zₛₚ), p∈ 1,2, . . . ,P and a safety distance sdₓ, sd_{y}, sd_{z} to obtain a safety boundary Saₚ=(xₛₐₚ, yₛₐₚ, zₛₐₚ), p∈1, 2, . . . , P.

S011: for coordinates of each focal point in (xᵢ′,y^{j}′,zₖ′) obtaining, through the following calculation, new coordinate points corresponding to the limit positions of the focal region boundary on X, Y and Z axes:

$$( {x}_{i}^{′} \pm \frac{{WX}_{0}}{2} , {y}_{j}^{′} , {z}_{k}^{′} ) , ( {x}_{i}^{′} , {y}_{j}^{′} \pm \frac{{WY}_{0}}{2} , {z}_{k}^{′} ) , ( {x}_{i}^{′} , {y}_{j}^{′} , {z}_{k}^{′} \pm \frac{{L}_{k}}{2} ) .$$

S012: further determining by the ray method whether the new coordinate points are within the safety boundary Saₚ=(xₛₐₚ, yₛₐₚ,zₛₐₚ) and if any new coordinate point is not within the safety boundary, then deleting the corresponding focal point coordinates of the new coordinate point in (xᵢ′,y^{j}′,zₖ′).

S013: repeating S010 and S011 until the coordinates of each focal point in (xᵢ′,y^{j}′,zₖ′) are traversed and final target coordinates (xᵢ′,y^{j}′,zₖ′) are obtained.

In this embodiment, the central control unit 1 performs intelligent planning of target emission parameters in the target region according to different focal region sizes when the focal point is in different spatial positions. The target emission parameters include focal region sound power Pᵤₙ, n=1,2, . . . ,N, where N is the number of final target coordinates, emission power P_{em,n} of each array element, emission signal voltage U_{m,n} and emission signal duty cycle D_{m,n}, m=1,2, . . . ,M, where M is the number of array elements in the phased array transducer.

As shown in FIG. 6, the method for intelligent planning of target emission parameters comprises the following steps:

S101: calculating focal region sound power Pᵤₙ of different focal point coordinates according to focal region length Lₖ of target coordinates, focal region width WXₖ of X axis, focal region width WYₖ of Y axis and set spatial peak time average sound intensity Iₛₚₜₐ:

$${P}_{un} = {I}_{spta} \times π \times {( 0.25 ( {WX}_{k} + {WY}_{k} ) )}^{2} ;$$

setting different sound power according to the actual focal region area when the focal point is in different spatial positions can realize consistency of the spatial peak time average sound intensity Iₛₚₜₐ of the focal region when the focal region area is different.

S102: calculating emission power P_{em,n} of each array element at different focal point coordinates according to the power weight Qₘ and electroacoustic conversion efficiency Kₘ of each array element of the phased array transducer:

$${P}_{em , n} = \frac{{P}_{un}}{{K}_{m} {\sum }_{m = 1}^{M} {Q}_{m}} , m = 1 , 2 , … , M ; n = 1 , 2 , … , N .$$

For the phased array transducer, the directivity angle of each array element is different in different focusing positions, so when calculating the electric power needed to be emitted by different array elements of the phased array transducer, the power weights of different array elements are considered, which is conducive to higher focusing efficiency and precise control of ultrasonic irradiation energy. In addition, the electroacoustic conversion efficiency varies with array elements. The set electric power is calculated according to different electroacoustic conversion efficiencies of the array elements, which is also conducive to the precise control of ultrasonic irradiation energy.

In this embodiment, power weight Qₘ and electroacoustic conversion efficiency Kₘ are measured on each array element of the phased array transducer by using a sound power meter and a sound field test system.

S103: calculating emission signal voltage (U_{m,n}) of each array element at different focal point coordinates according to the impedance characteristic (Zₘ),m=1,2, . . . ,M of each array element of the phased array transducer:

$${U}_{m , n} = \sqrt{\frac{{P}_{em , n} {Z}_{m}}{\frac{B}{C}}} , m = 1 , 2 , … , M ; n = 1 , 2 , … , N ;$$

where, C is the number of scanning lines needed by the ultrasonic imaging unit to scan a frame of ultrasonic image. In this embodiment, C is 128, and B is 64.

In this embodiment, impedance characteristic (Zₘ), m=1,2, . . . ,M is measured on each array element of the phased array transducer by using an impedance analyzer.

S104: calculating the maximum value of (U_{m,n}) recording it as U₀, and setting U₀ as a supply voltage of a power amplifier circuit in the phased array emission unit 3.

S105: calculating emission signal duty cycle D_{m,n} of each array element of the phased array transducer according to U_{m,n} and U₀:

$${D}_{m , n} = 0.5 \sqrt{\frac{{U}_{m , n}}{{U}_{0}}} .$$

Further, the central control unit 1 has a temperature measurement algorithm based on ultrasonic radio frequency data, which can calculate the temperature rise in the focal region according to the image data and application data. When the temperature rise in the focal region exceeds the safe temperature threshold, the central control unit 1 stops emitting ultrasound and sends a reminding signal to the operator.

The temperature measurement algorithm based on ultrasonic radio frequency data has been disclosed in patent ZL201710876349.5 “Ultrasonic Method for Measuring Biological Tissue Temperature Change based on Thermal Expansion and Gating Algorithm”.

Above the basic principle, main features and advantages of the present invention are described. Those of ordinary skill in the art should understand that the above embodiments do not in any way limit the scope of protection of the present invention, and that any technical solution obtained by means of equivalent replacement, etc. falls within the scope of protection of the present invention.

The parts not covered by the present invention are identical to or can be implemented using the prior art.

1. An ultrasonic phased array system based on a method for intelligent planning of target parameters, comprising a central control unit, an ultrasonic imaging unit, a phased array emission unit, a mechanical motion unit, a degassed water treatment unit and a composite probe, wherein:
the central control unit comprises one or more processors, which are used for controlling the ultrasonic imaging unit, the phased array emission unit, the mechanical motion unit, the degassed water treatment unit and the composite probe and for planning target coordinates and emission parameters in a target region;
the ultrasonic imaging unit acquiring image data of the target region through an ultrasonic imaging probe in the composite probe;
the phased array emission unit generating one or more focal points in the target region by independently controlling emission phases of different array elements of a phased array transducer and being able to control focusing positions of one or more focal points;
the mechanical motion unit comprising a mechanical motion driver and a multi-dimensional motion mechanical structure, and being used for moving the composite probe;
the degassed water treatment unit being used for generating degassed water, transmitting the degassed water to a water tank of the composite probe and controlling the degassed water to circulate between the water tank and the degassed water treatment unit; and
the composite probe comprising a phased array transducer, an ultrasonic imaging probe and an information storage device.

2. The ultrasonic phased array system based on a method for intelligent planning of target parameters according to claim 1, wherein the central control unit receives the following information set by a user: spatial peak time average sound intensity Iₛₚₜₐ, ultrasonic irradiation time t and temperature threshold Tₚ of the focal region.

3. The ultrasonic phased array system based on a method for intelligent planning of target parameters according to claim 1, wherein the image data acquired by the ultrasonic imaging unit includes grayscale image data and color image data; at the same time, ultrasonic RF data of the target region being also acquired, which is an original ultrasonic echo signal after beamforming; the ultrasonic imaging unit sending a frame pulse signal to the phased array emission unit when a first line of each frame of ultrasonic image starts scanning, and sending a line pulse signal to the phased array emission unit when each line of each frame of ultrasonic image except the first line starts scanning.

4. The ultrasonic phased array system based on a method for intelligent planning of target parameters according to claim 1, wherein the phased array emission unit controls the phased array transducer to emit ultrasound and counts line pulse signals sent by the ultrasonic imaging unit when receiving a frame pulse signal from the ultrasonic imaging unit; stops the phased array transducer sending ultrasound and counts line pulse signals again when the count reaches a first set threshold A; continues to control the phased array transducer to emit ultrasound when the count reaches a second set threshold B; repeats the above process when receiving a frame pulse signal from the ultrasonic imaging unit again; by controlling the first set threshold and the second set threshold, the phased array transducer being stopped emitting ultrasound when the ultrasonic imaging unit scans ultrasonic images in the target region, thereby avoiding the interference of high-intensity ultrasound on weak ultrasonic echo signals in the target region.

5. The ultrasonic phased array system based on a method for intelligent planning of target parameters according to claim 1, wherein the information storage device is used for storing various parameters of the phased array transducer;
the phased array transducer consisting of two or more independent array elements; and
the ultrasonic imaging probe being coaxially assembled with the center of the phased array transducer, and the ultrasonic imaging probe being a 2D ultrasonic imaging probe or a 3D ultrasonic imaging probe.

6. The ultrasonic phased array system based on a method for intelligent planning of target parameters according to claim 1, wherein the ultrasonic imaging probe is a 2D ultrasonic imaging probe, and the composite probe further comprising a position control device; the position control device controlling the 2D ultrasonic imaging probe to rotate along the central axis according to the position of focal point coordinates, and having the plane where the imaging field of the 2D ultrasonic imaging probe is located pass through the focal points coordinates all the time so as to realize real-time display of a focal position through an ultrasonic image in the process of ultrasonic emission.

7. The ultrasonic phased array system based on a method for intelligent planning of target parameters according to claim 6, wherein when the target coordinates are (xᵢ, yⱼ,zₖ) the position control device controls the ultrasonic imaging probe to rotate along the central axis at an angle of a, which is calculated according to the following formula:
$$\{ \begin{matrix}α = arctan ( \frac{{y}_{j}}{{x}_{i}} ) , {x}_{i} > 0 , {y}_{j} > 0 \\ α = π + arctan ( \frac{{y}_{j}}{{x}_{i}} ) , {x}_{i} < 0 \\ α = 2 π + arctan ( \frac{{y}_{j}}{{x}_{i}} ) , {x}_{i} > 0 , {y}_{j} < 0\end{matrix} .$$

8. The ultrasonic phased array system based on a method for intelligent planning of target parameters according to claim 1, wherein the central control unit performs intelligent planning of target coordinates in the target region according to different focal region sizes when the focal point is in different spatial positions; the method for intelligent planning of target coordinates in the target region comprising the following steps:
S001: setting the center of the top surface of the phased array transducer as an origin of a coordinate system, the axis direction of the phased array transducer as Z axis, the imaging scanning direction of the imaging probe as X axis, and the direction perpendicular to the imaging scanning direction of the imaging probe as Y axis;
S002: obtaining a coordinate set of pixels of the target region boundary set by a user;
S003: calculating according to the coordinate set of pixels and the distance between pixels dₚᵢₓₑₗ to obtain: physical coordinates of the target region boundary Sₚ=(xₛₚ,yₛₚ, zₛₚ),p∈1, 2, . . . ,P;
S004: obtaining the following built-in information of the system: safety distance sdₓsd_{y} sd_{z} between focal region boundary and target region boundary in X, Y, Z axis;
obtaining the following information set by the user: distance dₓ, d_{y}, d_{z} between focal region boundaries in X, Y, Z direction;
obtaining the following information set by the user: spatial peak time average sound intensity Iₛₚₜₐ, ultrasonic irradiation time t and temperature threshold Tₚ of the focal region;
S005: expanding the target region boundary Sₚ into a rectangular area Sₚ;
wherein, an upper surface and a lower surface of the rectangular area Sₚ′ are perpendicular to Z axis and pass through points (0,0, min(zₛₚ)) and (0,0,max(zₛₚ)) respectively; a left surface and a right surface being perpendicular to X axis and passing through points (min(xₛₚ),0,0) and (max(xₛₚ),0,0) respectively; and a front surface and rear surface being perpendicular to Y axis and passing through points (0,min(yₛₚ),0) and (0, max(yₛₚ),0) respectively;
S006: setting target coordinates within the rectangular area Sₚ′ to:
(xᵢ′,yⱼ′,zₖ′),i∈1,2, . . . ,1;j∈1,2, . . . ,J;k∈1,2, . . . ,K;
where, the point (xᵢ′,yⱼ′, zₖ′) is located in the X-Y plane which passes through a point (0,0, zₖ′) and is perpendicular to Z axis;
S007: calculating: coordinates (zₖ′),k ∈ 1, 2, . . . , K of the target (xᵢ′,yⱼ′, zₖ′) on Z axis, i.e., the Z axis coordinates that a different X-Y plane where the target is located passes, coordinates (xᵢ′)i ∈1,2, . . . , I of the target (xᵢ′,yⱼ′, zₖ′) on X axis, and coordinates (yⱼ′), j∈1, 2, . . . , J of the target (xᵢ′,yⱼ′, zₖ′) on Y axis;
S008: calculating according to a target region boundary Sₚ=(xₛₚ,yₛₚ,zₛₚ), p∈1,2, . . . ,P and a safety distance sdₓ, sd_{y}, sd_{z} to obtain a safety boundary Saₚ=(xₛₐₚ,yₛₐₚ,zₛₐₚ), p∈1, 2, . . . , P
S009: for coordinates of each focal point in (xᵢ′,yⱼ′, zₖ′) obtaining, through the following calculation, new coordinate points corresponding to the limit positions of the focal region boundary on X, Y and Z axes:
$$( {x}_{i}^{′} \pm \frac{{WX}_{0}}{2} , {y}_{j}^{′} , {z}_{k}^{′} ) , ( {x}_{i}^{′} , {y}_{j}^{′} \pm \frac{{WY}_{0}}{2} , {z}_{k}^{′} ) , ( {x}_{i}^{′} , {y}_{j}^{′} , {z}_{k}^{′} \pm \frac{{L}_{k}}{2} ) ;$$
S010: further determining by the ray method whether the new coordinate points are within the safety boundary saₚ=(xₛₐₚ, yₛₐₚ, zₛₐₚ) and if any new coordinate point is not within the safety boundary, then deleting the corresponding focal point coordinates of the new coordinate point in (xᵢ′,yⱼ′, zₖ′); and
S011: repeating S009 and S010 until the coordinates of each focal point in (xᵢ′,yⱼ′, zₖ′) are traversed and final target coordinates (xᵢ,yⱼ, zₖ) are obtained.

9. The ultrasonic phased array system based on a method for intelligent planning of target parameters according to claim 8, wherein calculating coordinates (zₖ′),k∈1,2, . . . , K of the target (xᵢ′,yⱼ′, zₖ′) on Z axis comprises the following steps:
a) calculating first coordinates z₁′: z₁′=min(zₛₚ)+d_{z}+Δ z×n, where n≥1, n is an integer; n is added with 1 step by step, starting from 1, to perform the following calculation:
calculating focal region length L, focal region width WX of X axis, and focal region width WY of Y axis by the focal region calculation method of sound field when the focal point is located at (0,0, min(zₛₚ)+d_{z}+Δ z×n), recording min(zₛₚ)+d_{z}+Δ z×n as z₁′ when Formula
$$\frac{L}{2} \geq Δ z \times n$$
is satisfied for the first time, i.e., coordinates on Z axis that the first X-Y plane where the target is located passes, and now recording the focal region length as L₁ focal region width of X axis as WX₁, and focal region width of Y axis as WY₁;
where, Δz is a step threshold when the coordinates of the target on Z axis are calculated;
b) calculating m^{th} coordinates zₘ′:
$${z}_{m}^{′} = {z}_{m - 1}^{′} + \frac{{L}_{m - 1}}{2} + {d}_{z} + Δ z \times n ,$$
where n≥1, n is an integer, m≥2, m is an integer; n is added with 1 step by step, starting from 1, to perform the following calculation:
calculating focal region length L, focal region width WX of X axis, and focal region width WY of Y axis by the focal region calculation method of sound field when the focal point is located at
$$( 0 , 0 , {z}_{m - 1}^{′} + \frac{{L}_{m - 1}}{2} + {d}_{z} + Δ z \times n ) ,$$
recording
$${z}_{m - 1}^{′} + \frac{{L}_{m - 1}}{2} + {d}_{z} + Δ z \times n$$
as zₘ′ when Formula
$$\frac{{L}_{m}}{2} \geq Δ z \times n$$
is satisfied for the first time, and
$$\frac{{L}_{m}}{2} \leq ( max ( {z}_{sp} ) - {d}_{z} ) ,$$
i.e., coordinates on Z axis that the m^{th} X-Y plane where the target is located passes, and now recording the focal region length as Lₘ, focal region width of X axis as WXₘ, and focal region width of Y axis as WYₘ;
c) adding 1 to m, then repeating step b), stopping calculation when
$$\frac{{L}_{m}}{2} > ( max ( {z}_{sp} ) - {d}_{z} )$$
appears for the first time, recording the result
$${z}_{m - 1}^{′} + \frac{{L}_{m - 1}}{2} + {d}_{z} + Δ z \times n$$
of the calculation before this calculation as z_{K}′, i.e., coordinates on Z axis that the K^{th} X-Y plane where the target is located passes, and now recording the focal region length as L_{K}, focal region width of X axis as WX_{K}, and focal region width of Y axis as WY_{K}, where K=m−1; and
d) obtaining from the above step the coordinates (zₖ′) of the target (xᵢ′,yⱼ′, zₖ′) on Z axis, focal region length (Lₖ), focal region width (WXₖ) of X axis, and focal region width (WYₖ) of Y axis, k∈1,2, . . . ,K.

10. The ultrasonic phased array system based on a method for intelligent planning of target parameters according to claim 8, wherein calculating coordinates (xᵢ′)i ∈1,2, . . . , I of the target (xᵢ′,yⱼ′, zₖ′) on X axis comprises the following steps:
a) calculating I′ according to Formula min(xₛₚ)+dₓ×I′+WX₀×I′+dₓ≤max(xₛₚ) and recording the integer part of I′ as I; and
b) calculating xᵢ′ according to the following formula:
$${x}_{i}^{′} = min ( {x}_{sp} ) + {d}_{x} \times i + {WX}_{0} \times i - \frac{W {X}_{0}}{2} , i ∈ 1 , 2 , … , I .$$

11. The ultrasonic phased array system based on a method for intelligent planning of target parameters according to claim 8, wherein calculating coordinates (yⱼ′), j∈1,2, . . . , J of the target (xᵢ′,yⱼ′, zₖ′) on Y axis comprises the following steps:
a) calculating J′ according to Formula min(yₛₚ)+d_{y}×J′+WY₀×d_{y}≤max (yₛₚ), and recording the integer part of J′ as J; and
b) calculating yⱼ′ according to the following formula:
$${y}_{j}^{′} = min ( {y}_{sp} ) + {d}_{y} \times j + W {Y}_{0} \times j - \frac{{WY}_{0}}{2} , j ∈ 1 , 2 , … , J .$$

12. The ultrasonic phased array system based on a method for intelligent planning of target parameters according to claim 1, wherein, the central control unit performs intelligent planning of target emission parameters in the target region according to different focal region sizes when the focal point is in different spatial positions; the method for intelligent planning of target emission parameters comprising the following steps:
S101: calculating focal region sound power Pᵤₙ of different focal point coordinates according to focal region length Lₖ of target coordinates, focal region width WXₖ of X axis, focal region width WYₖ of Y axis and set spatial peak time average sound intensity Iₛₚₜₐ:
$${P}_{u n} = {I}_{spta} \times π \times {( 0.25 ( {WX}_{k} + {WY}_{k} ) )}^{2} ;$$
S102: calculating emission power P_{em,n} of each array element at different focal point coordinates according to the power weight Qₘ and electroacoustic conversion efficiency Kₘ of each array element of the phased array transducer:
$${P}_{em , n} = \frac{{P}_{u n}}{{K}_{m} {\sum }_{m = 1}^{M} {Q}_{m}} , m = 1 , 2 , … , M ; n = 1 , 2 , … , N ;$$
S103: calculating emission signal voltage (U_{m,n}) of each array element at different focal point coordinates according to the impedance characteristic (Zₘ),m=1,2, . . . ,M of each array element of the phased array transducer
$${U}_{m , n} = \sqrt{\frac{{P}_{em , n} {Z}_{m}}{\frac{B}{C}}} , m = 1 , 2 , … , M ; n = 1 , 2 , … , N ;$$
where, C is the number of scanning lines needed by the ultrasonic imaging unit to scan a frame of ultrasonic image;
S104: calculating the maximum value of (U_{m,n}), recording it as U₀, and setting U₀ as a supply voltage of a power amplifier circuit in the phased array emission unit; and
S105: calculating emission signal duty cycle D_{m,n} of each array element of the phased array transducer according to U_{m,n} and U₀:
$${D}_{m , n} = 0.5 \sqrt{\frac{{U}_{m , n}}{{U}_{0}}} .$$
